(19) United States
(12) Patent Application Publication
(10) Pub. No.: US 2017/0305877 A1
(43) Pub. Date: Oct. 26, 2017

(54) Processes for the Preparation of Rotigotine and Intermediates Thereof

(71) Applicant: Apotex Inc., Toronto (CA)

(72) Inventors: Michael B. Johansen, Cambridge (CA); Probal Kanti Datta, Hamilton (CA); Yajun Zhao, Brantford (CA); Gamini Weeratunga, Ancaster (CA)

(21) Appl. No.: 15/513,859

(22) PCT Filed: Sep. 23, 2015
(86) PCT No.: PCT/CA2015/000502

§ 371 Date: Mar. 23, 2017

(30) Foreign Application Priority Data
Sep. 24, 2014 (CA) 62/054966

(51) Int. Cl. C07D 333/20; C07C 213/02; C07C 213/08; C07C 217/74

(52) U.S. Cl. CPC C07D 333/20; C07C 217/74; C07C 213/02; C07C 213/08

(57) ABSTRACT

The present invention provides processes for the preparation of a compound of Formula 2 or a salt thereof, wherein R¹ is selected from the group consisting of H, C₁-C₃ alkyl, and C(0)R³; R³ is selected from the group consisting of C₁-C₆ alkyl, C₆-C₁₀aryl and C₇-C₂₀ arylalkyl; the carbon atom marked with “*” is racemic, enantiomerically enriched in the (R)-configuration, or enantiomerically enriched in the (S)-configuration. Also provided are intermediate compounds of the processes.

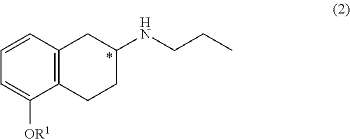

### TECHNICAL FIELD

The present invention relates to the field of synthesis of organic compounds and, in particular, to syntheses of Rotigotine and intermediates thereof.

### BACKGROUND

Rotigotine, the common name for (6S)-5,6,7,8-tetrahydro-6-[propyl[2-(2-thienyl)ethyl]amino]-1-naphthalenol, is indicated for the treatment of idiopathic Parkinson's disease. It is represented by the structure of Formula 1:

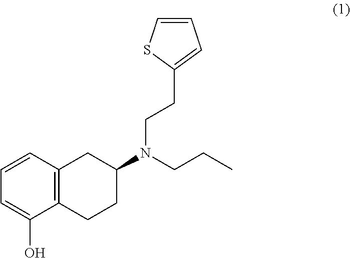 [SMILES: CCCN(CCc1cccs1)[C@H]1CCc2c(O)cccc2C1]

U.S. Pat. No. 4,885,308 A provides a method for treating the symptoms of parkinsonism which comprises administering to a human or other mammal suffering from the symptoms of parkinsonism an effective amount of a compound selected from the group consisting of optically-active or racemic compounds represented by the general formula:

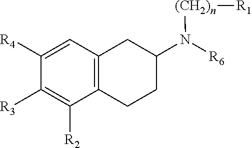

wherein R₁, R₂, R₃, R₄ and R₆ are defined therein. Preferably R₂ is oxygen. Preferably, R₂ is OA and A is H, and the compound is the (−)-isomer.

WO 01/038321 A1 relates to a process for preparing optically active and racemic nitrogen-substituted 2-aminotetralins of Formula:

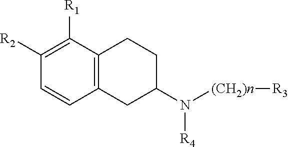

wherein R₁ is OA; R₂ is selected from the group consisting of H and OA; A is H or is selected from the group consisting of a straight or a branched alkyl chain having from 1 to 3 carbon atoms, (a), (b), (c) and (d),

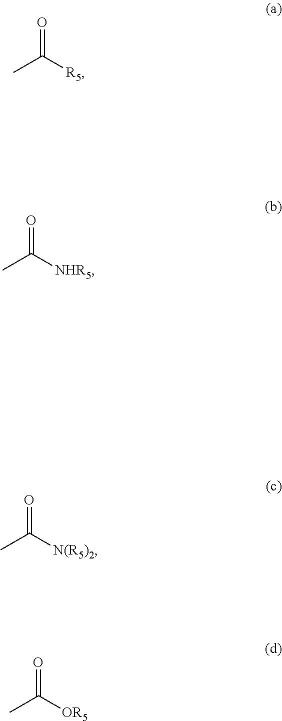

wherein R₅ is selected from the group consisting of C₁-C₂₀ alkyl, C₆-C₁₀ aryl and C₇-C₂₀ arylalkyl; R₃ is selected from the group consisting of alkoxy, cycloalkoxy, optionally substituted phenyl, 3-pyridyl, 4-pyridyl, (e), (f) and (g),

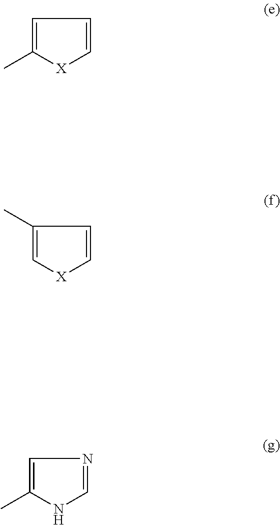

where X is S, O or NH: R₄ is an unbranched alkyl chain having from 1 to 3 carbon atoms; and n is an integer from 1 to 5, wherein the process comprises alkylating the corresponding unsubstituted 2-aminotetralin of Formula:

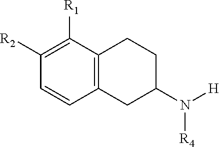

with a reactant of Formula Z—(CH₂)ₙ—R₃; wherein R₃ and n are as defined above and Z is a leaving group, in the presence of a base, wherein the base is selected from the group consisting of alkali metal carbonate and alkali metal bicarbonate, and wherein the amount of the base is less than about a 1.9-fold molar excess with respect to the amount of the 2-aminotetralin.

WO 2009/056791 A1 relates to the preparation of N,N-disubstituted aminotetralins, such as Rotigotine, as the free base form or as a pharmaceutically acceptable salt.

In JP2002-187875 A, 5-methoxy-2-tetralone is reductively aminated with ammonia in a proper solvent in the presence of a catalytic reduction catalyst to give a racemic isomer of 2-amino-5-methoxytetralin. The racemic isomer is subjected to optical resolution in a proper solvent by using a proper optically active organic acid.

WO 2010/035111 A1 provides a process for the preparation of Rotigotine and of pharmaceutically acceptable salts thereof, which comprises the reductive amination of an amine of Formula:

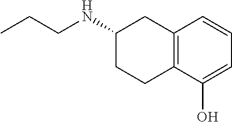

with a 2-thienylacetic acid-sodium boron hydride complex and which makes use of hydrobromide:

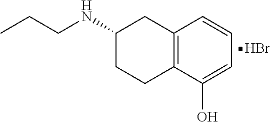

as an intermediate. Furthermore, two novel crystalline forms are disclosed.

WO 2010/043571 A1 describes a process for the preparation of optically active (S)-(−)-2-(N-propylamino)-5-methoxytetraline and (S)-(−)-2-(N-propylamino)-5-hydroxytetraline compounds based on the optical resolution of mixtures of the enantiomers of 2-(N-propylamino)-5-methoxytetraline and 2-(N-propylamino)-5-hydroxytetraline, respectively. This process comprises reacting a mixture of the enantiomers of said compounds with an optically active organic acid to form diastereoisomeric salts and separating the salts by crystallization. Said compounds are useful in the preparation of (6S)-(−)-5,6,7,8-tetrahydro-6-[propyl-(2-thienyl)ethyl]amino-1-naphthol (Rotigotine).

WO 2010/066755 A1 describes a process for the preparation of (6S)-(−)-5,6,7,8-tetrahydro-6-[propyl-(2-thienyl)ethyl]amino-1-naphthol (Rotigotine) comprising: (a) acetylating (S)-(−)-5-hydroxy-N-n-propyl-2-amino tetraline to afford the acetate; (b) reacting this acetate, (−)-5-acetoxy-N-n-propyl-2-aminotetraline, with 2-(2-thienyl)ethanol-2-nitrobenzenesulfonate; (c) hydrolyzing (6S)-(−)-1-acetoxy-5,6,7,8-tetrahydro-6-[propyl-(2-thienyl) ethyl]amino-1-naphthalene to afford (6S)-(−)-5,6,7,8-tetrahydro-6-[propyl-(2-thienyl)ethyl]amino-1-naphthol (Rotigotine) and (d) purifying Rotigotine either by the acetylation reaction and subsequent hydrolysis of the formed acetate or by salification of Rotigotine through hydrochloride or hydrobromide formation and subsequent base release.

WO 2010/073124 A2 provides processes for the preparation of (−)-(S)-5-hydroxy-2-[N-n-propyl-N-2-(2-thienyl)ethylamino]tetralin (Rotigotine) or a pharmaceutically acceptable salt thereof. Provided further herein is a highly pure Rotigotine or a pharmaceutically acceptable salt thereof substantially free of impurities, processes for the preparation thereof, and pharmaceutical compositions comprising highly pure Rotigotine or a pharmaceutically acceptable salt thereof substantially free of impurities.

IN 2009CH00795 relates to an improved process for preparation of Rotigotine or an acid addition salt thereof. The present invention also relates to a process for the preparation of crystalline Rotigotine hydrochloride and crystalline Rotigotine base. The present invention further relates to crystalline (−)-5-hydroxy-N-n-propyl-2-aminotetralin, a key intermediate used in the preparation of Rotigotine.

CN 101717392 A discloses a method for preparation of the compound of Formula:

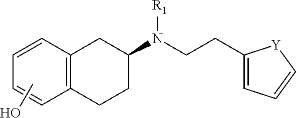

or its pharmaceutically acceptable salts thereof. The method comprises the steps of: (1) using a compound of the Formula:

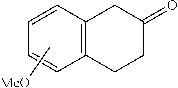

as starting material and carrying out a reductive amination with a suitable reducing agent to obtain a compound of the Formula:

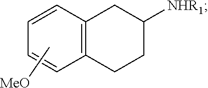

(2) reacting 2-quinary heterocyclic substituted ethanol with appropriate reagent to obtain a compound of the Formula:

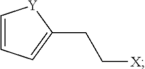

(3) after carrying out chiral separation on the compound of the Formula:

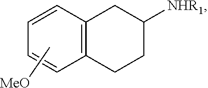

it is condensed with the compound of the Formula:

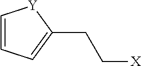

under an alkaline condition to obtain a compound of the Formula:

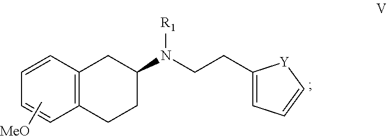

and (4) carrying out demethylation on the compound under the condition of appropriate temperature and solvent to obtain the compound of the Formula:

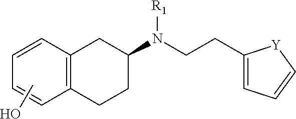

wherein R₁ in each Formula is selected from C1-8 alkyl groups or aromatic bases which can be arbitrarily substituted, X is selected from halogen atoms or p-toluenesulfonic acid groups and methanesulfonic acid groups for protecting alcoholic extract hydroxyl groups, and Y is selected from O, S and N.

U.S. Pat. No. 8,614,337 B2 discloses chiral compound S-5-substituted-N-2′-(thienyl-2-yl-)ethyl-tetralin-2-amine or its chiral acid salts and preparation method thereof, and the method for preparing Rotigotine by using the chiral compound. Racemic 5-substituted-N-2′-(thien-2-yl-)ethyl-tetralin-2-amine is resolved by using a conventional chiral acid to obtain an optically pure chiral acid salt of S-5-substituted-N-2′-(thien-2-yl)ethyl-tetralin-2-amine, which is then dissociated to obtain S-5-substituted-N-2′-(thien-2-yl)ethyl-tetralin-2-amine. The compound S-5-substituted-N-2′-(thien-2-yl)ethyl-tetralin-2-amine or a chiral acid salt thereof is alkylated and deprotected to produce Rotigotine.

WO 2011/095539 A2 provides an alternative synthesis of N-substituted aminotetralines which synthesis comprises catalytic asymmetric hydrogenation of compounds of general Formula (A):

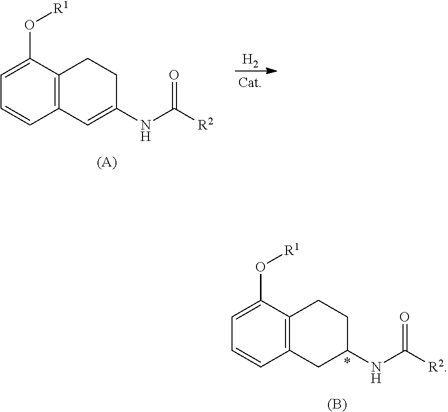

WO 2011/146610 A2 discloses methods of preparing (S)—N-(1,2,3,4-tetrahydronaphthalen-2-yl)propionamide compounds from N-(3,4-dihydronaphthalen-2-yl)propionamide compounds:

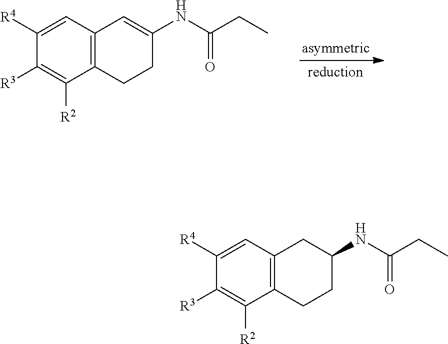

where the constituent variables are as defined.

WO 2011/161255 A2 discloses an alternative synthesis of N-substituted aminotetralines comprising resolution of N-substituted aminotetralins of Formula:

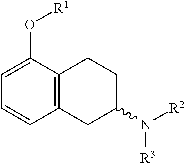

wherein R¹, R² and R³ are as defined for a compound of Formula:

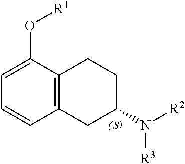

CN 102731326 A relates to the field of pharmaceutical chemistry, and discloses a synthetic method for preparation of (S)-amino-5-methoxy-1, 2, 3, 4-tetrahydronaphthalene hydrochloride. The method of the invention comprises the following steps: (1) carrying out an addition-elimination reaction on 5-methoxy-2-tetralone and R-(+)-α-phenylethylamine to generate a compound shown as Formula:

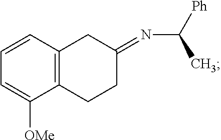 [SMILES: COc1cccc2c1CC/C(=N\[C@H](C)c1ccccc1)C2]

(2) reducing the compound under the action of a reducing agent to produce a compound shown as Formula:

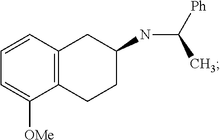 [SMILES: COc1cccc2c1CC[C@H](N[C@H](C)c1ccccc1)C2]

and (3) reacting the compound with a salt forming agent to produce a hydrochloride of the compound, and then carrying out a reduction reaction under the action of a palladium carbon catalyst to generate the (S)-amino-5-methoxy-1, 2, 3, 4-tetrahydronaphthalene hydrochloride.

U.S. Pat. No. 8,809,590 B2 discloses a method for industrially preparing a nitrogen substituted 6-amino-5,6,7,8-tetrahydronaphthol. The method comprises reacting a nitrogen substituted amino-5,6,7,8-tetrahydronaphthol compound of Formula:

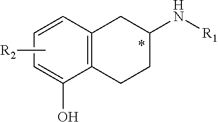

with a 2-substituted ethyl sulfonate compound of Formula:

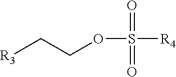

under an alkaline condition and in the presence of a sulfite.

CN 103058985 A discloses a process for preparing Rotigotine based on 2-amino-5-methoxynaphthalene. The process comprises the steps of splitting to obtain (S)-2-amino-5-methoxynaphthalene by L-tartaric acid, reacting (S)-2-amino-5-methoxynaphthalene with bromopropane to obtain (S)-2-(N-n-propyl)amido-5-methoxynaphthalene; reacting (S)-2-(N-n-propyl)amido-5-methoxy naphthalene with 2-(2-bromethyl) thiophene to obtain (S)-2-[N-propyl-N-[2-(2-thiophene) ethyl]amino]-5-methoxyl-1,2,3,4-tetrahydronaphthalene, and removing methyl under the effect of hydrobromic acid to obtain a target product Rotigotine (−)-(S)-2-[N-propyl-N-[2-(2-thiophene)ethyl]amino]-5-hydroxy-1,2,3,4-tetrahydro naphthalene, wherein the purity is higher than 99%.

US 2014/0046095 A1 discloses a method of preparing (S)-2-amino-5-methoxytetralin hydrochloride [(S)-2-amino-5-methoxyl-1,2,3,4-tetrahydronaphthalene hydrochloride], comprising the steps of: (1) producing a compound:

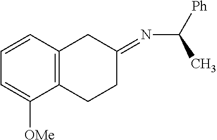 [SMILES: COc1cccc2c1CC/C(=N\[C@H](C)c1ccccc1)C2]

by an addition-elimination reaction of 5-methoxy-2-tetralone and R-(+)-α-phenylethylamine; (2) producing a compound:

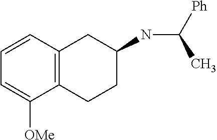 [SMILES: COc1cccc2c1CC[C@H](N[C@H](C)c1ccccc1)C2]

by a reduction reaction of the compound:

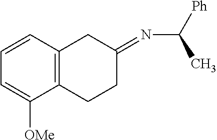 [SMILES: COc1cccc2c1CC/C(=N\[C@H](C)c1ccccc1)C2]

with a reducing agent; and (3) producing a hydrochloride by reacting the compound:

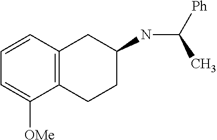 [SMILES: COc1cccc2c1CC[C@H](N[C@H](C)c1ccccc1)C2]

with a salt-forming agent, then carrying out reduction reaction with a palladium-carbon catalyst to produce (S)-2-amino-5-methoxytetralin hydrochloride.

McDermed, J. D. et al. in J. Med. Chem. 1976, 19(4), 547-549 disclose, in an effort to identify further the structural requirements for central dopamine receptor agonists, some monohydroxyl analogs of the known agonist 5,6-dihydroxy-2-dipropylamino-1,2,3,4-tetrahydronaphthalene were synthesized. They were examined for production of emesis in dogs and stereotyped behavior in rats. The most potent was 5-hydroxy-2-dipropylamino-1,2,3,4-tetrahydro naphthalene, which was more potent than apomorphine but less so than the dihydroxyl analog. The two enantiomers of the monohydroxyl analog were synthesized by conventional methods from an optically active intermediate, 2-benzylamino-5-methoxy-1,2,3,4-tetrahydronaphthalene. The resolution of this amine was performed with the aid of mandelic acid. Dopaminergic activity was found to be confined to the levo enantiomer. Requirements for both substitution and chirality in the tetralins were found to correspond closely to those known for the dopaminergic aporphines.

Horn, A. S. et al. in Pharmaceutisch Weekblad Sci. Ed. 1985, 7, 208-211 disclose the synthesis of a new potent and selective D2 dopamine receptor agonist N-0437 of the 2-aminotetralin group. The results of a radioreceptor binding assay using a homogenate of porcine anterior pituitary as a tissue source for D₂ dopamine receptors and ³H-spiperone as radioligand demonstrate that this compound is one of the most potent compounds so far evaluated in this test system.

Cusack, J. N. et al. in Drugs Fut. 1993, 18(11), 1005-1008 disclose N-0923, a Dopamine D2 Agonist.

Wikstrom, H. et al. in J. Med. Chem. 1985, 28, 215-225 disclose a detailed structure-activity relationship for resolved, centrally acting dopamine (DA) agonists acting on both pre- and postsynaptic DA receptors. The compounds resolved are 5- and 7-hydroxy-2-(di-n-propylamino)tetralin and cis- and trans-7-hydroxy-4-n-propyl-1,2,3,4,4a,5,6,10b-octahydrobenzo quino[f]quinoline.

Seiler, M. P. et al. in J. Med. Chem. 1986, 29, 912-917 disclose 5-hydroxy-2-aminotetralin derivatives in which one N-alkyl substituent carries a functional group have been prepared and their dopaminergic activities compared with those of 5-hydroxy-2-(di-n-propylamino)tetralin (5-OH-DPAT) and known ergolines. Several members of the series demonstrated high affinities in dopamine (DA) receptor binding and DA agonist properties in the rotational behavior model in the range of known potent ergolines. The results suggest that the accessory binding site for the larger N-alkyl substituent of the 5-hydroxy-2-aminotetralins can accommodate various neutral and bulky functionalities and is probably identical with the site(s) to which the 8-substituents of the ergolines bind.

Hirayama, Y. et al. in Org. Process. Res. Dev. 2005, 9, 30-38 disclose an expeditious synthesis of (S)-2-amino-5-methoxytetralin via resolution.

### SUMMARY

The present invention is based at least in part, on processes for the preparation of a compound of Formula 2:

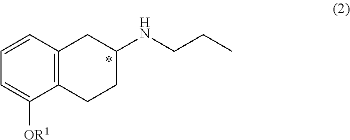

or a salt thereof as well as intermediate compounds within such processes.

Illustrative embodiments of the present invention provide a process for the preparation of a compound of Formula 2:

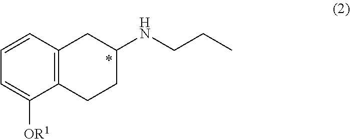

or a salt thereof, the process comprising hydrogenating, in the presence of a catalyst, a compound of Formula 3:

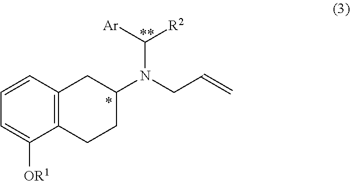

wherein R¹ is selected from the group consisting of: H, C₁-C₃ alkyl, and

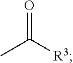

R² is selected from the group consisting of: H and C₁-C₃ alkyl; R³ is selected from the group consisting of: C₁-C₆ alkyl, C₆-C₁₀ aryl and C₇-C₂₀ arylalkyl; Ar is selected from the group consisting of: Ar-aryl and substituted Ar-aryl; the carbon atom marked with “*” is racemic, enantiomerically enriched in the (R)-configuration, or enantiomerically enriched in the (S)-configuration; and when R² is not H, the carbon atom marked with “**” is racemic, enantiomerically enriched in the (R)-configuration, or enantiomerically enriched in the (S)-configuration.

Illustrative embodiments of the present invention provide a process described herein wherein the catalyst is selected from the group consisting of: palladium, platinum and Raney™ Nickel.

Illustrative embodiments of the present invention provide a process described herein wherein the catalyst is selected from the group consisting of: palladium hydroxide on carbon and palladium on carbon.

Illustrative embodiments of the present invention provide a process described herein further comprising isolating an intermediate compound of Formula 8:

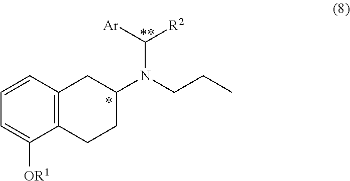

or a salt thereof during the hydrogenating.

Illustrative embodiments of the present invention provide a process described herein further comprising reacting a compound of Formula 4:

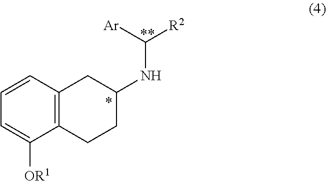

with a compound of Formula 5:

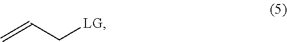

thereby forming the compound of Formula 3, wherein LG is a leaving group.

Illustrative embodiments of the present invention provide a process described herein wherein the leaving group is selected from the group consisting of: bromide, iodide, sulfonyloxy groups and carbonates.

Illustrative embodiments of the present invention provide a process described herein further comprising reductive amination of a compound of Formula 6:

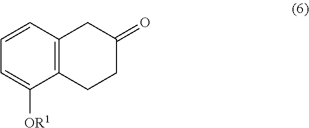

with a compound of Formula 7:

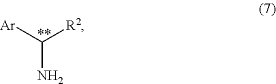

thereby forming the compound of Formula 4.

Illustrative embodiments of the present invention provide a process described herein further comprising isolating an intermediate compound of Formula 9:

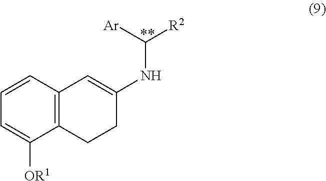

or a salt thereof.

Illustrative embodiments of the present invention provide a process described herein wherein the reductive amination is conducted with a hydride reducing agent selected from the group consisting of: sodium borohydride, potassium borohydride, lithium borohydride, sodium cyanoborohydride and sodium triacetoxyborohydride.

Illustrative embodiments of the present invention provide a process described herein wherein R¹ is C₁-C₃ alkyl; R² is methyl; Ar is selected from the group consisting of: naphthyl, phenyl, and substituted phenyl; the carbon atom marked with “*” is enriched in the (S)-configuration; and the carbon atom marked with “**” is enriched in the (R)-configuration.

Illustrative embodiments of the present invention provide a process described herein wherein the compound of Formula 3 is selected from the group consisting of:

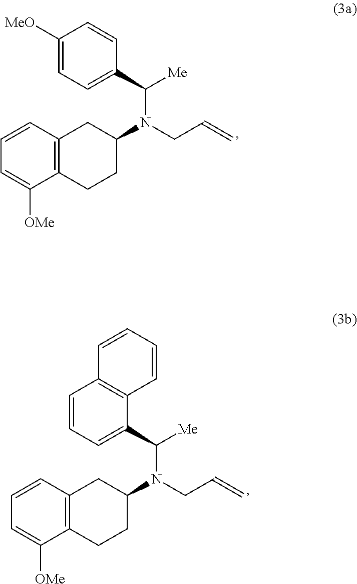 [SMILES: C=CCN([C@H]1CCc2c(cccc2OC)C1)[C@H](C)c1ccc(OC)cc1 ; C=CCN([C@H]1CCc2c(cccc2OC)C1)[C@H](C)c1cccc2ccccc12]

and salts thereof.

Illustrative embodiments of the present invention provide a process for the preparation of a compound of Formula 4:

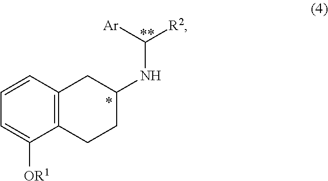

and salts thereof, comprising reductive amination of a compound of Formula 6:

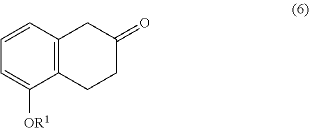

with a compound of Formula 7:

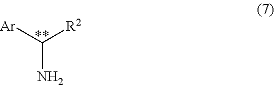

wherein R¹ is selected from the group consisting of: H, C₁-C₃ alkyl, and

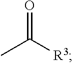

R² is C₁-C₃ alkyl; R³ is selected from the group consisting of: C₁-C₆ alkyl, C₆-C₁₀ aryl and C₇-C₂₀ arylalkyl; Ar is selected from the group consisting of: naphthyl and substituted phenyl; the carbon atom marked with “*” is enantiomerically enriched in the (R)-configuration, or enantiomerically enriched in the (S)-configuration; and the carbon atom marked with “**” is enantiomerically enriched in the (R)-configuration, or enantiomerically enriched in the (S)-configuration.

Illustrative embodiments of the present invention provide a process described herein further comprising isolating an intermediate compound of Formula 9:

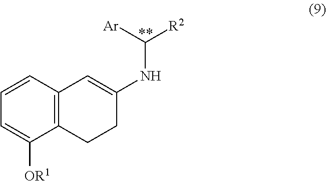

or a salt thereof.

Illustrative embodiments of the present invention provide a process described herein wherein the reductive amination is conducted with a hydride reducing agent selected from the group consisting of: sodium borohydride, potassium borohydride, lithium borohydride, sodium cyanoborohydride and sodium triacetoxyborohydride.

Illustrative embodiments of the present invention provide a process described herein wherein the compound of Formula 4 is selected from the group consisting of:

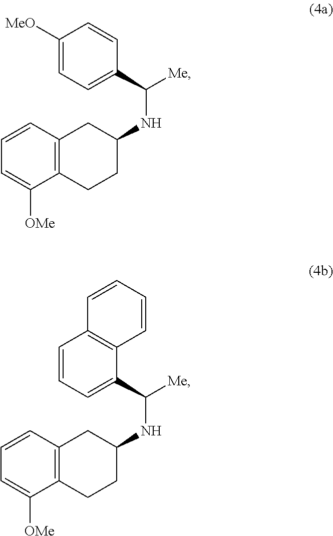 [SMILES: COc1ccc([C@@H](C)N[C@H]2CCc3c(cccc3OC)C2)cc1 ; COc1cccc2c1CC[C@H](N[C@H](C)c1cccc3ccccc13)C2]

and salts thereof.

Illustrative embodiments of the present invention provide a compound of Formula 3:

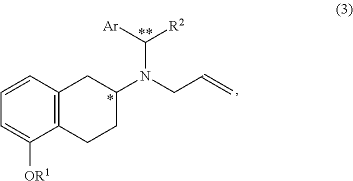

or a salt thereof wherein R¹ is selected from the group consisting of: H, C₁-C₃ alkyl, and

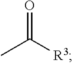

R² is selected from the group consisting of: H and C₁-C₃ alkyl; R³ is selected from the group consisting of: C₁-C₆ alkyl, C₆-C₁₀ aryl and C₇-C₂₀ arylalkyl; Ar is selected from the group consisting of: Ar-aryl and substituted Ar-aryl; the carbon atom marked with “*” is racemic, enantiomerically enriched in the (R)-configuration, or enantiomerically enriched in the (S)-configuration; and when R² is not H, the carbon atom marked with “**” is racemic, enantiomerically enriched in the (R)-configuration, or enantiomerically enriched in the (S)-configuration.

Illustrative embodiments of the present invention provide a compound selected from the group consisting of:

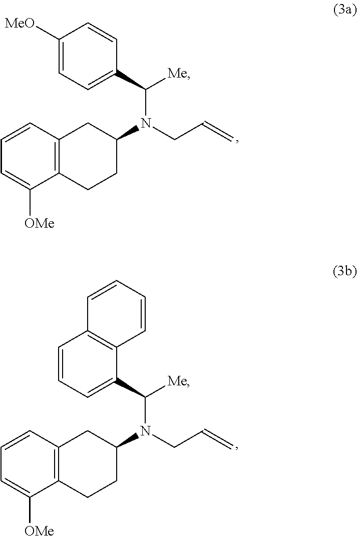 [SMILES: C=CCN([C@H]1CCc2c(cccc2OC)C1)[C@H](C)c1ccc(OC)cc1 ; C=CCN([C@H]1CCc2c(cccc2OC)C1)[C@H](C)c1cccc2ccccc12]

and salts thereof.

Illustrative embodiments of the present invention provide a compound selected from the group consisting of:

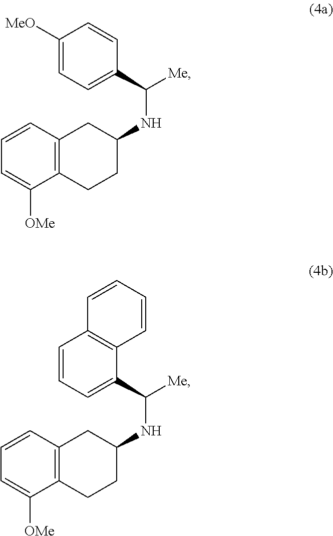 [SMILES: COc1ccc([C@@H](C)N[C@H]2CCc3c(cccc3OC)C2)cc1 ; COc1cccc2c1CC[C@H](N[C@H](C)c1cccc3ccccc13)C2]

and salts thereof.

Illustrative embodiments of the present invention provide a compound selected from the group consisting of:

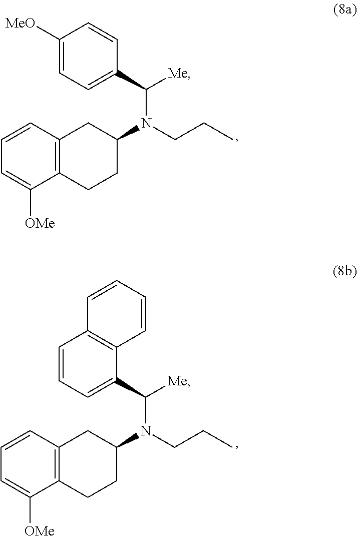 [SMILES: CCCN([C@H]1CCc2c(cccc2OC)C1)[C@H](C)c1ccc(OC)cc1 ; CCCN([C@H]1CCc2c(cccc2OC)C1)[C@H](C)c1cccc2ccccc12]

and salts thereof.

Illustrative embodiments of the present invention provide a compound selected from the group consisting of:

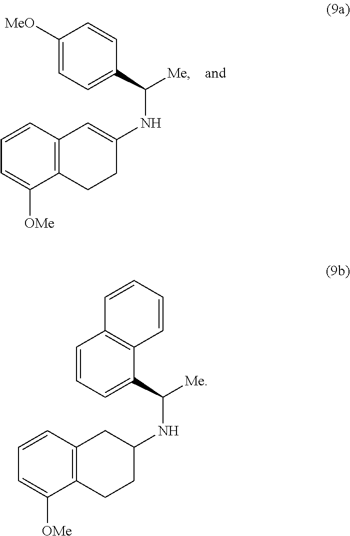 [SMILES: COc1ccc([C@@H](C)NC2=Cc3cccc(OC)c3CC2)cc1 ; "and" ; COc1cccc2c1CCC(N[C@H](C)c1cccc3ccccc13)C2]

Illustrative embodiments of the present invention provide a process described herein further comprising converting the compound of Formula 2 or a salt thereof to Rotigotine or a pharmaceutically acceptable salt thereof.

Other aspects and features of the present invention will become apparent to those ordinarily skilled in the art upon review of the following description of specific embodiments of the invention in conjunction with the accompanying figures.

### DETAILED DESCRIPTION

As used herein, the term “alkyl” by itself or as part of another substituent, means, unless otherwise stated, a straight or branched chain, hydrocarbon radical, having the number of carbon atoms designated (e.g. C₁-C₁₀ or 1- to 10-membered means one to ten carbons). Examples of saturated hydrocarbon radicals include, but are not limited to, groups such as methyl, ethyl, n-propyl, isopropyl, n-butyl, t-butyl, isobutyl, sec-butyl, homologs and isomers of, for example, n-pentyl, n-hexyl and the like.

As used herein the term “leaving group” refers to a group that can be displaced by nucleophiles. Examples of leaving groups include halogen atoms (for example, chlorine, bromine and iodine), sulfonyloxy groups (for example, methanesulfonyloxy, trifluoromethanesulfonyloxy, p-toluenesulfonyloxy) and carbonates.

As used herein, the term “aryl” by itself or as part of another substituent, means, unless otherwise stated, a polyunsaturated, aromatic, hydrocarbon substituent which can be a single ring or multiple rings (often from 1 to 3 rings) which are fused together or linked covalently. Non-limiting examples of aryl groups include: phenyl, 2-naphthyl and 4-biphenyl. The term “arylalkyl” is meant to include those radicals in which an aryl group is attached to an alkyl group (for example, benzyl, phenylethyl, etc.). An exception to this definition occurs with respect to the term “Ar-aryl”. In the case of “Ar-aryl”, the group must also be a group that provides a benzylic position at the carbon atom marked with an “**” in a compound of Formula 3 that is cleavable by hydrogenation, but is otherwise an aryl group as defined previously.

As used herein, the term “hydrogenation” or “hydrogenating” means a chemical reaction that results in addition of two hydrogen atoms. As such, it is meant to include both addition of hydrogen to saturate an unsaturated bond as well as addition of hydrogen to a single bond to cause bond breakage (hydrogenolysis) as well as other reactions involving the addition of two hydrogen atoms.

As used herein, the term “substituted” refers to the replacement of a hydrogen atom on a compound with a substituent group. A substituent may be a non-hydrogen atom or multiple atoms of which at least one is a non-hydrogen atom and one or more may or may not be hydrogen atoms. For example, without limitation, substituted compounds may comprise one or more substituent's selected from the group consisting of: R″, OR″, NR″R″, SR″, halogen, OC(O)R″, C(O)R″, CO₂R″, CONR″R′″, NR″C(O)₂R″, S(O)R″, S(O)₂R″, CN, and NO₂. As used herein, each R″, R′″, and R″″ may be selected, independently, from the group consisting of: aryl, alkyl and arylalkyl groups. Examples include methyl and methoxy.

As used herein, the term “diastereomeric ratio” refers to the ratio of the percentage of one diastereoisomer in a mixture to that of the other.

As used herein, the term “Pd(OH)₂/C” means palladium hydroxide on carbon and the term “Pd/C” means palladium on carbon.

An embodiment of the present invention provides a process for the preparation of a compound of Formula 2:

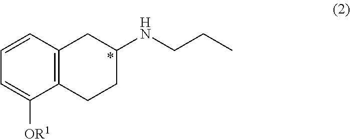

or a salt thereof, comprising catalytic hydrogenation of a compound of Formula 3:

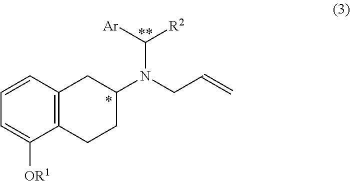

wherein

R¹ is selected from the group consisting of: H, C₁-C₃ alkyl, and

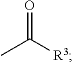

R² is selected from the group consisting of: H and C₁-C₃ alkyl;

R³ is selected from the group consisting of: C₁-C₆ alkyl, C₆-C₁₀ aryl and C₇-C₂₀ arylalkyl;

Ar is selected from the group consisting of: Ar-aryl and substituted Ar-aryl;

the carbon atom marked with “*” can be racemic, or enantiomerically enriched in the (R)- or (S)-configuration; and

when R² is not H, the carbon atom marked with “**” can be racemic, or enantiomerically enriched in the (R)- or (S)-configuration.

In some embodiments of the compound of Formula 3, R¹ is C₁-C₃ alkyl; R² is C₁-C₃ alkyl; Ar is selected from the group consisting of: naphthyl, phenyl, and substituted phenyl and the carbon atoms marked with “*” and “**” are enantiomerically enriched in the (R)- or (S)-configuration.

In many embodiments of the compound of Formula 3, R¹ is C₁-C₃ alkyl; R² is methyl; Ar is selected from the group consisting of: naphthyl, phenyl, and substituted phenyl; the carbon atom marked with “*” is enriched in the (S)-configuration and the carbon atom marked with “**” is enriched in the (R)-configuration.

The hydrogenation catalyst may be selected from the group consisting of: palladium, platinum, rhodium, ruthenium, and Raney™ nickel. The hydrogenation catalyst may be finely dispersed solids or adsorbed on an inert support such as carbon, alumina or barium sulfate. The hydrogenation catalyst may be wet or dry. The catalyst loading may be from about 0.1 wt % palladium with respect to the weight of the compound of Formula 3 and/or Formula 8. The catalyst loading may be from about 0.2 wt % to about 0.8 wt % palladium with respect to the weight of the compound of Formula 3 and/or 8.

The hydrogenation may be conducted in a first solvent. The first solvent may be selected from the group consisting of: alcohols (for example, methanol, isopropanol), ethers (for example, methyl t-butyl ether, tetrahydrofuran), chlorinated hydrocarbons (for example, dichloromethane), alkyl esters (for example, ethyl acetate, isopropyl acetate), aromatic hydrocarbons (for example, toluene), hydrocarbons (for example, cyclohexane), ketones (for example, methylisobutyl ketone), water and mixtures thereof.

The hydrogenation may be conducted at a suitable temperature from about 10° C. to about the boiling point of the solvent. Often the hydrogenation may be conducted at a temperature from about 20° C. to about 70° C.

The hydrogenation may be conducted at atmospheric pressure or at a higher pressure in a suitable vessel. The reaction may be conducted under a pressure from about 80 psi to about 150 psi.

The hydrogenation may be conducted in the presence of an acid, particularly the hydrogenative cleavage of the benzylic group. A suitable acid may be an acid which has a lower pka than the conjugate acid of the amine position. Suitable acids may include weak acids (for example, acetic acid, formic acid), strong mineral acids (for example, hydrochloric acid, phosphoric acid, sulfuric acid) and strong organic acids (for example, alkyl and aryl sulphonic acids).

The hydrogenation may be performed in a stepwise manner, wherein a compound of Formula 8:

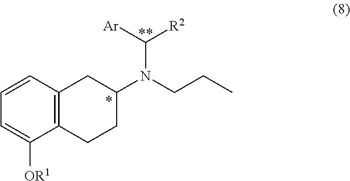

or a salt thereof is generated and isolated prior to further cleavage by hydrogenation to yield the compound of Formula 2. In such embodiments:

R¹ is selected from the group consisting of: H, C₁-C₃ alkyl, and

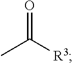

R² is selected from the group consisting of: H and C₁-C₃ alkyl;

R³ is selected from the group consisting of: C₁-C₆ alkyl, C₆-C₁₀ aryl and C₇-C₂₀ arylalkyl;

Ar is selected from the group consisting of: Ar-aryl and substituted Ar-aryl;

the carbon atom marked with “*” can be racemic, or enantiomerically enriched in the (R)- or (S)-configuration; and

when R² is not H, the carbon atom marked with “**” can be racemic, or enantiomerically enriched in the (R)- or (S)-configuration.

In some embodiments of the compound of Formula 8, R¹ is C₁-C₃ alkyl; R² is C₁-C₃ alkyl; Ar is selected from the group consisting of: naphthyl, phenyl, and substituted phenyl and the carbon atoms marked with “*” and “**” are enantiomerically enriched in the (R)- or (S)-configuration.

In many embodiments of the compound of Formula 8, R¹ is C₁-C₃ alkyl; R² is methyl; Ar is selected from the group consisting of: naphthyl, phenyl, and substituted phenyl; the carbon atom marked with “*” is enriched in the (S)-configuration and the carbon atom marked with “**” is enriched in the (R)-configuration.

Alternatively, the hydrogenation may be performed as a one-pot procedure wherein the compound of Formula 2 or a salt thereof is obtained directly from the compound of Formula 3 without isolation of any intermediates.

The hydrogenation conditions described herein may be applied to a stepwise process or to a one-pot process and the conditions applied for hydrogenation of each of the two positions of the compound of Formula 3 may be the same or different.

In some embodiments, in either the stepwise or one-pot procedure, a first hydrogenation may form an intermediate compound of Formula 8 and a second hydrogenation may form the product of Formula 2. In either the stepwise or the one-pot procedure, conditions for the first hydrogenation may be milder than the conditions for the second hydrogenation.

In some embodiments, in either the stepwise or one-pot procedure, the first hydrogenation to form the compound of Formula 8 may be conducted in the absence of acid at ambient pressures and with lower activity catalysts such as palladium on barium sulphate. A preferred solvent for the first hydrogenation may be a non-polar hydrocarbon (for example, heptanes).

In some embodiments, in either the stepwise or one-pot procedure, the second hydrogenation to form the compound of Formula 2 may be conducted in the presence of acid at higher pressures and with higher activity catalysts such as palladium on carbon. A preferred solvent for the second hydrogenation may be a polar solvent (for example, alcohols).

In some embodiments, a process is provided wherein the compound of Formula 3 is prepared by a process comprising reaction of a compound of Formula 4:

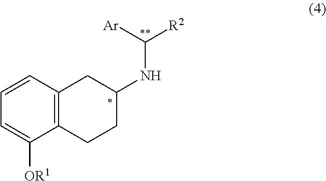

with a compound of Formula 5:

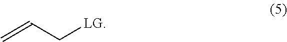

In such embodiments:

R¹ is selected from the group consisting of: H, C₁-C₃ alkyl, and

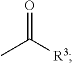

R² is selected from the group consisting of: H and C₁-C₃ alkyl;

R³ is selected from the group consisting of: C₁-C₆ alkyl, C₆-C₁₀ aryl and C₇-C₂₀ arylalkyl;

Ar is selected from the group consisting of: Ar-aryl and substituted Ar-aryl;

the carbon atom marked with “*” can be racemic, or enantiomerically enriched in the (R)- or (S)-configuration;

when R² is not H, the carbon atom marked with “**” can be racemic, or enantiomerically enriched in the (R)- or (S)-configuration; and

LG is a leaving group.

In some embodiments of the compound of Formula 5, the leaving groups is a halogen atom, a sulfonyloxy group, or a carbonate. In some embodiments of the compound of Formula 5, the leaving group is chlorine, bromine, iodine, methanesulfonyloxy, trifluoromethanesulfonyloxy, or p-toluenesulfonyloxy.

In some embodiments of the compound of Formula 4, R¹ is C₁-C₃ alkyl; R² is C₁-C₃ alkyl; Ar is selected from the group consisting of: naphthyl, phenyl, and substituted phenyl and the carbon atoms marked with “*” and “**” are enantiomerically enriched in the (R)- or (S)-configuration.

In many embodiments of the compound of Formula 4, R¹ is C₁-C₃ alkyl; R² is methyl; Ar is selected from the group consisting of: naphthyl, phenyl, and substituted phenyl; the carbon atom marked with “*” is enriched in the (S)-configuration and the carbon atom marked with “**” is enriched in the (R)-configuration.

In some embodiments, when the compound of Formula 4 is a compound 4a or 4b:

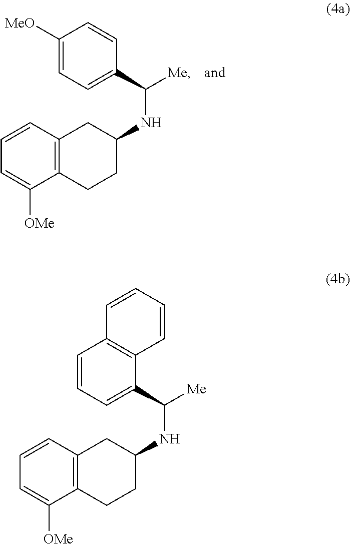 [SMILES: COc1ccc([C@@H](C)N[C@H]2CCc3c(cccc3OC)C2)cc1 ; COc1cccc2c1CC[C@H](N[C@H](C)c1cccc3ccccc13)C2]

propylation using a propylating agent such as propyl bromide under standard SN2 conditions shows poor conversion to the compound of Formula 8. In the present invention, the compound of the Formula 5 behaves as a propyl ‘surrogate’ and this provides improved conversion. Without being bound by theory, it is believed that steric hindrance around the amine position is the possible cause of the poor conversion when standard alkylation conditions are utilized. The mechanism is believed to be that the propyl surrogate is attacked by an amine at the carbon of the double bond most removed from the leaving group. The amine is bulky and can better approach this more remote position than the carbon directly attached to the leaving group. With the more standard alkylation conditions, it is believed that the bulky amine would need to attack at the carbon directly attached to the leaving group, which is believed to be hindered and may proceed poorly, if at all with a standard propyl-LG reagent. Nevertheless, regardless of the mechanism, use of a propyl surrogate provides a desired outcome.

The reaction of the compound of Formula 4 and the compound of Formula 5 may be conducted in the presence of a suitable base. The suitable base may be inorganic or organic. The inorganic base may be selected from the group consisting of: metal carbonates, phosphates, metal hydroxides, metal alkoxides. Organic bases may be selected from the group consisting of: tertiary amines (for example, triethylamine, diisopropylethylamine) and amidines (for example, DBU).

The reaction of the compound of Formula 4 and the compound of Formula 5 may be conducted in a second solvent. The second solvent may be selected from the group consisting of: nitriles (for example, acetonitrile, proprionitrile), N,N-dialkylamides (for example, N,N-dimethylformamide, N,N-dimethylacetamide), aromatic hydrocarbons (for example, toluene, benzene, chlorobenzene), alkyl acetates (for example, ethyl acetate, isopropyl acetate, isobutyl acetate), ketones (for example, acetone, methyl isobutyl ketone), sulfoxides (for example, dimethylsulfoxide), sulfones (for example, sulfolane) and pyrrolidines (for example, N-methyl pyrrolidine).

The compound of Formula 5 may be used as such or it may be generated insitu. For example, catalytic or more than stoichiometric amounts of sodium iodide, other metal iodides or other sources of iodide may be used in combination with allyl bromide or other compound of Formula 5 to generate an allyl iodide in situ.

The reaction of the compound of Formula 4 and the compound of Formula 5 may be conducted a temperature from about 20° C. Often, the temperature is from about 70° C. to about 130° C. Due to the lower boiling point of some embodiments of Formula 5, sealed conditions may be required depending upon the reaction temperature employed.

In some embodiments, a process is provided wherein the compound of Formula 4 is prepared by a process comprising reductive amination of a compound of Formula 6:

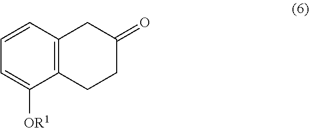

with a compound of Formula 7:

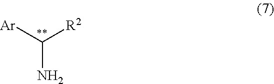

wherein

R¹ is selected from the group consisting of: H, C₁-C₃ alkyl, and

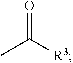

R² is selected from the group consisting of: H and C₁-C₃ alkyl;

R³ is selected from the group consisting of: C₁-C₆ alkyl, C₆-C₁₀ aryl and C₇-C₂₀ arylalkyl;

Ar is selected from the group consisting of: Ar-aryl and substituted Ar-aryl; and,

when R² is not H, the carbon atom marked with “**” can be racemic, or enantiomerically enriched in the (R)- or (S)-configuration.

In some embodiments of the compound of Formula 6, R¹ is C₁-C₃ alkyl;

In many embodiments of the compound of Formula 7, R² is C₁-C₃ alkyl; Ar is selected from the group consisting of: naphthyl, phenyl, and substituted phenyl and the carbon atom marked with “**” is enantiomerically enriched in the (R)- or (S)-configuration.

In some embodiments of the compound of Formula 7, R² is methyl; Ar is selected from the group consisting of: naphthyl, phenyl, and substituted phenyl; and the carbon atom marked with “**” is enriched in the (R)-configuration.

The reductive amination may be performed in a stepwise manner, wherein an intermediate compound of Formula 9:

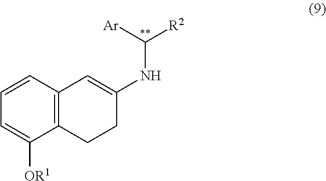

or a salt thereof is isolated. In such embodiments:

R¹ is selected from the group consisting of: H, C₁-C₃ alkyl, and

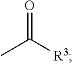

R² is selected from the group consisting of: H and C₁-C₃ alkyl;

R³ is selected from the group consisting of: C₁-C₆ alkyl, C₆-C₁₀ aryl and C₇-C₂₀ arylalkyl;

Ar is selected from the group consisting of: Ar-aryl and substituted Ar-aryl; and

when R² is not H, the carbon atom marked with “**” can be racemic, or enantiomerically enriched in the (R)- or (S)-configuration.

In some embodiments of the compound of Formula 9, R¹ is C₁-C₃ alkyl; R² is C₁-C₃ alkyl; Ar is selected from the group consisting of: naphthyl, phenyl, and substituted phenyl and the carbon atom marked with “**” is enantiomerically enriched in the (R)- or (S)-configuration.

In many embodiments of the compound of Formula 9, R¹ is C₁-C₃ alkyl; R² is methyl; Ar is selected from the group consisting of: naphthyl, phenyl, and substituted phenyl; the carbon atom marked with “*” is enriched in the (S)-configuration and the carbon atom marked with “**” is enriched in the (R)-configuration.

Alternatively, the reductive amination may be performed as a one-pot procedure wherein the compound of Formula 4 or a salt thereof is obtained directly without isolation of any intermediates.

The first step of the reductive amination may be treatment of the compound of Formula 6 with the compound of Formula 7. The reaction of the compounds of Formula 6 and Formula 7 may be conducted in a third solvent. The third solvent may be selected from the group consisting of: alcohols (for example, methanol, ethanol, isopropanol, n-propanol), ethers (for example, tetrahydrofuran), chlorinated hydrocarbons (for example, dichloromethane, dichloroethane), aromatic hydrocarbons (for example, toluene, benzene, chlorobenzene), alkyl acetates (for example, ethyl acetate, isopropyl acetate, isobutyl acetate), sulfoxides (for example, dimethylsulfoxide), sulfones (for example, sulfolane) and pyrrolidines (for example, N-methyl pyrrolidine). A mechanism for removal of generated water formed during the course of the reaction may be employed, such as a Dean-Stark apparatus or other appropriate drying agent (for example, sodium or magnesium sulfate, molecular sieves, titanium tetrachloride). The temperature may be from about 20° C. to about the boiling point of the solvent. The product of this step may or may not be isolated.

Following consumption of the starting materials, the intermediate product may be treated with a reducing agent to form the compound of Formula 4. The reducing agent may be selected from the group consisting of: borohydrides (for example, alkali metal borohydrides, sodium cyanoborohydride, sodium triacetoxyborohydride, zinc borohydride, tetraalkylammonium borohydride) and aluminum hydrides (for example, lithium aluminum hydride, diisobutylaluminum hydride). The reduction may be conducted in a fourth solvent. In some embodiments, the fourth solvent may be selected from the group consisting of: alcohols (for example, methanol, ethanol, isopropanol and n-propanol) and ethers (for example, methyl t-butyl ether and tetrahydrofuran). The fourth solvent may be the same or different than the third solvent. Often, when the compound of Formula 4 wherein the carbon atoms marked with “*” and “**” are enantiomerically enriched is desired, the fourth solvent may comprise an alcohol. The reduction may be conducted at a temperature from about −20° C. to about the boiling point of the solvent. When an enantiomerically enriched compound of Formula 4 is desired, lower temperatures in this range are preferred. When alcoholic solvents are employed, the reaction rate may decrease significantly at less than −10° C.

In some embodiments, a milder reductant such as sodium cyanoborohydride or sodium triacetoxyborohydride may be combined directly with the compound of Formula 6 and the compound of Formula 7 to produce the compound of Formula 4.

The compound of Formula 4 wherein the carbon atom marked with “*” is enantiomerically enriched may be prepared by the reductive amination of the compound of Formula 6 with the compound of Formula 7 when the carbon atom marked with “**” in Formula 7 is likewise enantiomerically enriched. When the carbon atom marked with “**” is racemic in the compound of Formula 7, the compound of Formula 4 which is racemic will result.

In some embodiments, compounds 4a, 4b, 9a and 9b:

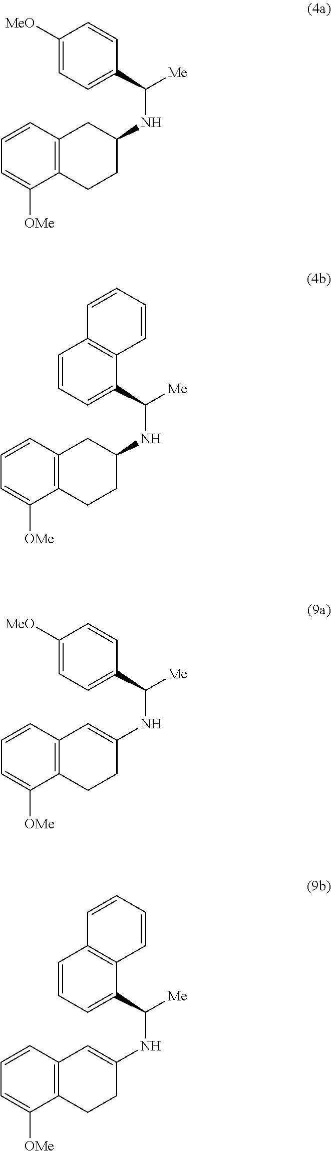 [SMILES: COc1ccc([C@@H](C)N[C@H]2CCc3c(cccc3OC)C2)cc1 ; COc1cccc2c1CC[C@H](N[C@H](C)c1cccc3ccccc13)C2 ; COc1ccc([C@@H](C)NC2=Cc3cccc(OC)c3CC2)cc1 ; COc1cccc2c1CCC(N[C@H](C)c1cccc3ccccc13)=C2]

are isolatable solids. Some other embodiments of Formula 4 and/or Formula 9 may be oils. Solid products may provide a superior ease of handling and an opportunity for additional chiral enrichment and purification compared to liquids and oily substances.

Compounds of Formula 3 wherein R² is H and the carbon atom marked with “*” is enriched in the (R)- or (S)-configuration may be prepared according to known procedures, for example, resolution procedures such as those described in McDermed, J. D. et al., J. Med. Chem. 1976, 19(4), 547-549.

### EXAMPLES

The following examples are illustrative of some of the embodiments of the invention described herein. These examples do not limit the spirit or scope of the invention in any way.

### Example 1 Preparation of Compound 9a

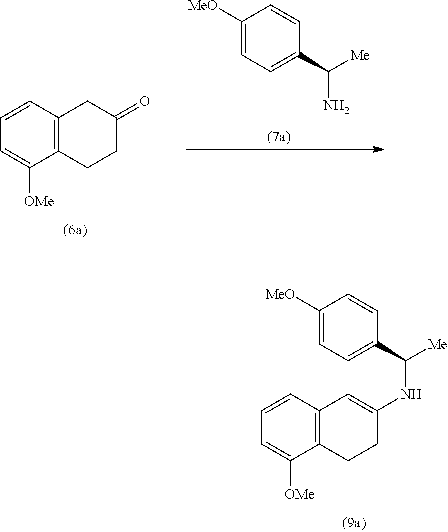 [SMILES: COc1cccc2c1CCC(=O)C2 ; COc1ccc([C@@H](C)N)cc1 ; COc1ccc([C@@H](C)NC2=Cc3cccc(OC)c3CC2)cc1]

A solution of 5-methoxy-2-tetralone (6a, 15 g, 85 mmoL) and (R)-(+)-1-(4-methoxyphenyl)ethylamine (7a, 13 g, 87 mmoL) in n-propanol (150 mL) was heated to reflux under Dean-Stark conditions, with a slow removal of 100 mL of the reaction solvent by distillation over 2 h. The mixture was then slowly cooled to 50° C., at which point precipitation was observed. The reaction mixture was further cooled to 0-5° C. over 4.5 h, then filtered. The collected precipitate was washed with cold n-propanol (3×15 mL) and dried under vacuum at room temperature to provide 9a as a yellowish, beige solid, 23 g, 86% yield. ¹H-NMR (400 MHz, CDCl₃): δ=7.25 (d, J=8.5 Hz, 2H), 6.86 (d, J=8.7 Hz, 2H), 6.41 (d, J=8.2 Hz, 1H), 6.21 (d, J=7.5 Hz, 1H), 5.86 (d, J=6.6 Hz, 2H), 4.80 (s, 1H), 4.38-4.32 (m, 1H), 3.71 (s, 3H), 3.69 (s, 3H), 2.68-2.52 (m, 1H), 2.50 (app s, 1H), 2.29-2.27 (m, 2H), 1.34 (d, J=6.8 Hz, 3H).

### Example 2 Preparation of Compound 9b

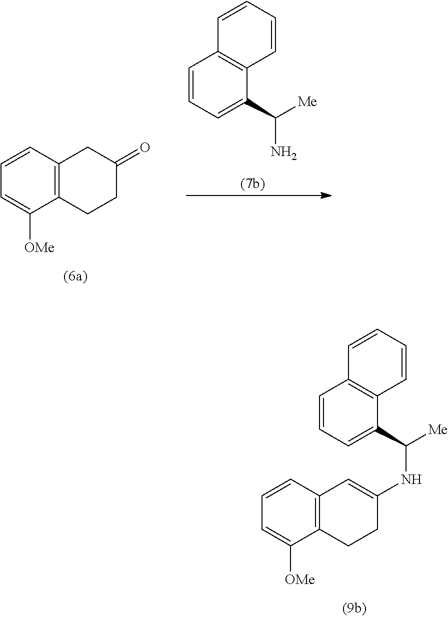 [SMILES: COc1cccc2c1CCC(=O)C2 ; C[C@@H](N)c1cccc2ccccc12 ; COc1cccc2c1CCC(N[C@H](C)c1cccc3ccccc13)=C2]

A solution of 5-methoxy-2-tetralone (6a, 15.0 g, 85 mmoL) and R-(+)-1-(1-naphthyl)ethylamine (7b, 15 g, 85 mmoL) in n-propanol (150 mL) was heated to reflux under Dean-Stark conditions, with a slow removal of 90 mL of the reaction solvent by distillation over 4 h. The mixture was then slowly cooled to 50° C., at which point precipitation was observed. The reaction mixture was further cooled to 0-5° C. over 2.5 h, then filtered. The collected precipitate was washed with cold n-propanol (2×15 mL) and dried under vacuum at room temperature to provide 9b as an on off-white solid, 26 g, 99% yield. ¹H-NMR (400 MHz, CDCl₃): δ=8.10 (d, J=8.3 Hz, 1H), 7.88 (d, J=7.8 Hz, 1H), 7.75 (d, J=8.1 Hz, 1H), 7.57-7.48 (m, 3H), 7.42 (dd, J=7.8, 7.5 Hz, 1H), 6.93 (dd, J=8.0, 7.9 Hz, 1H), 6.46 (d, J=8.1 Hz, 1H), 6.33 (d, J=7.6 Hz, 1H), 5.32-5.25 (m, 1H), 5.04 (s, 1H), 3.78 (s, 3H), 3.66 (d, J=5.2 Hz, 1H), 2.91-2.78 (m, 2H), 2.39-2.26 (m, 2H), 1.64 (d, J=6.7 Hz, 3H).

### Example 3 Preparation of Compound 4a

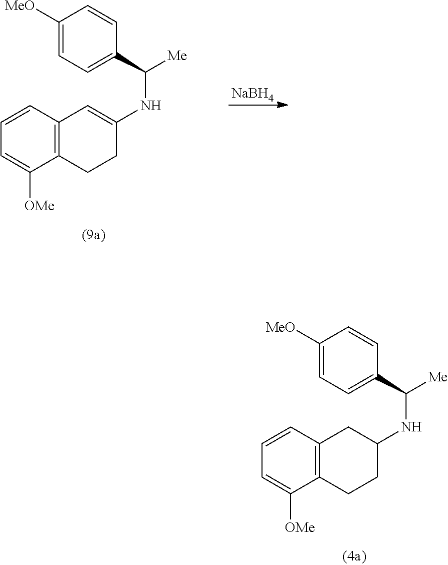 [SMILES: COc1ccc([C@@H](C)NC2=Cc3cccc(OC)c3CC2)cc1 ; "NaBH4" ; COc1ccc([C@@H](C)NC2CCc3c(cccc3OC)C2)cc1]

To a suspension of 9a (5.0 g, 16 mmoL) in 50 mL methanol at −5° C. to −2° C. was added sodium borohydride (0.9 g, 24 mmoL) in 5 portions at 20-30 min intervals over 2 h, maintaining the reaction internal temperature between −2° C. and −5° C. The reaction was maintained at −2° C. to −5° C. for an additional 1.5 h, at which point the reaction was determined to be complete by ¹H-NMR. The slurry was maintained at −2° C. to −5° C. for an additional 40 min then filtered. The collected solid was washed with cold methanol (3×5 mL) and dried under vacuum at room temperature to provide 4a as an on off-white solid, 3.4 g, 67% yield in >95:5 diastereomeric ratio, as determined by ¹H-NMR. ¹H-NMR (400 MHz, CDCl₃): δ=7.24 (d, J=9.8 Hz, 2H), 7.04 (dd, J=7.9, 7.8 Hz, 1H), 6.84 (d, J=8.5 Hz, 2H), 6.63 (dd, J=7.5, 4.1 Hz, 1H), 4.00 (q, J=6.5 Hz, 1H), 3.78 (app s, 6H), 2.88-2.74 (m, 3H), 2.58-2.44 (m, 2H), 2.13-2.09 (m, 1H), 1.57-1.45 (m, 1H), 1.34 (d, J=6.5 Hz, 3H).

### Example 4 Preparation of Compound 4a

A solution of 5-methoxy-2-tetralone (6a, 1.0 g, 5.7 mmoL) and (R)-(+)-1-(4-methoxyphenyl)ethylamine (7a, 0.9 g, 5.9 mmoL) in n-propanol (10 mL) was heated to reflux under Dean-Stark conditions, with a slow removal of 9 mL of the reaction solvent by distillation over 1 h. Methanol (18 mL) was then added and the slurry was cooled to 0° C. to −15° C. Sodium borohydride (0.25 g, 6.8 mmoL) was then added portionwise over 4 h. The reaction was then maintained at 0° C. to −10° C. for 2 h. The product was collected by filtration, washed with cold methanol (3×1 mL) and dried under vacuum to provide 4a as an on off-white solid, 0.89 g, 50% yield in >95:5 diastereomeric ratio, as determined by ¹H-NMR.

### Example 5 Preparation of Compound 4b

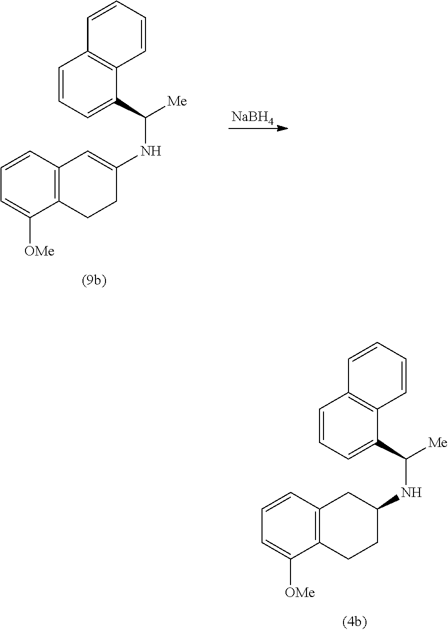 [SMILES: COc1cccc2c1CCC(N[C@H](C)c1cccc3ccccc13)=C2 ; "NaBH4" ; COc1cccc2c1CC[C@H](N[C@H](C)c1cccc3ccccc13)C2]

To a suspension of 9b (5.0 g, 15 mmoL) in methanol (50 mL) at 0-5° C. was added sodium borohydride (2.7 g, 70 mmoL) in portions (5×0.3 g) at 25-30 min intervals over 2.5 h followed by 2 portions of 0.6 g at the 5 h and 5.5 h marks, while maintaining the reaction internal temperature between 0° C. to 5° C. The reaction was maintain at 0-5° C. for an additional 17 h, at which point the reaction was determined to be complete by ¹H-NMR. The slurry was then filtered and the collected solid was washed with cold methanol (2×10 mL) and dried under vacuum at room temperature to provide 4b as a white solid, 4.0 g, 80% yield in 95:5 diastereomeric ratio, as determined by ¹H-NMR. ¹H-NMR (400 MHz, CDCl₃): δ=8.22 (d, J=8.3 Hz, 1H), 7.85 (d, J=7.8 Hz, 1H), 7.72 (d, J=8.1 Hz, 1H), 7.68 (d, J=7.1 Hz, 1H), 7.52-7.44 (m, 3H), 7.05 (dd, J=7.8, 7.8 Hz, 1H), 6.65 (d, J=7.7 Hz, 1H), 6.61 (d, J=8.1 Hz, 1H), 4.92-4.90 (m, 1H), 3.77 (s, 3H), 2.98-2.81 (m, 3H), 2.63 (dd, J=15.4, 8.4 Hz, 1H), 2.49-2.40 (m, 1H), 2.14-2.11 (m, 1H), 1.66-1.54 (m, 1H), 1.51 (d, J=6.4 Hz, 3H).

### Example 6 Preparation of Compound 4b

A solution of 5-methoxy-2-tetralone (6a, 4.5 g, 26 mmoL) and R-(+)-1-(1-naphthyl)ethylamine (7b, 4.5 g, 26 mmoL) in n-propanol (45 mL) was heated to reflux under Dean-Stark conditions, with a slow removal of 42 mL of the reaction solvent by distillation over 1 h. Methanol (70 mL) was added and the slurry was cooled to 0° C. to −2° C. Sodium borohydride (1.5 g, 38 mmoL) was then added portionwise over 3 h. The reaction was maintained at 0° C. to −2° C. for 16 h. A further portionwise addition of sodium borohydride (1.0 g, 26 mmoL) was completed over a 1 h period, followed by a maintain period of 1 h. The reaction was then distilled to 45 mL total volume, and cooled to 0-5° C. and maintained for 3 h. The product was collected by filtration, washed with cold methanol (2×10 mL) and dried under vacuum to provide 4b as an on off-white solid, 7.1 g, 84% yield in 93:7 diastereomeric ratio, as determined by ¹H-NMR.

### Example 7 Preparation of Compound 3a

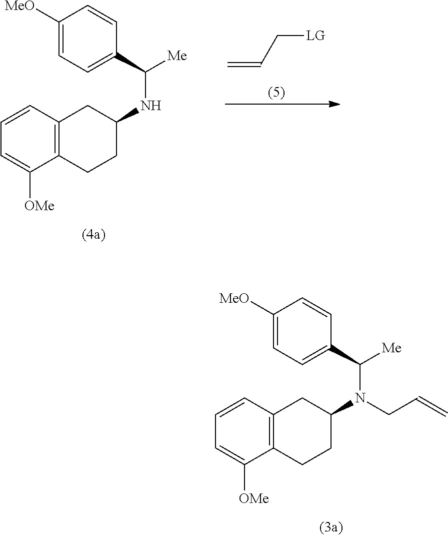

A sealed vessel was charged with 4a (0.82 g, 2.6 mmoL), sodium carbonate (0.35 g, 3.2 mmoL), allyl bromide (0.33 g, 2.6 mmoL) and acetonitrile (4 mL). The vessel was sealed under nitrogen and heated to 85° C. for 23 h, at which point the reaction was determined to be complete by TLC. The reaction was then cooled to 22° C., extracted into ethyl acetate with saturated aqueous potassium carbonate solution, then brine solution. The organic phase was dried over anhydrous sodium sulphate and then the solids were removed by filtration. The mother liquor was concentrated and the residue was purified by silica gel column chromatography (gradient elution, 5-10% ethyl acetate in heptanes) to provide 3a as a clear, light yellow viscous oil, 0.78 g, 84% yield. ¹H-NMR (400 MHz, CDCl₃): δ=7.28 (d, J=8.5 Hz, 2H), 7.06 (dd, J=7.9, 7.8 Hz, 1H), 6.82 (d, J=8.6 Hz, 2H), 6.67 (d, J=8.6 Hz, 2H), 6.61 (d, J=8.1 Hz, 1H), 5.92-5.84 (m, 1H), 5.20 (app dd, J=17.2, 1.3 Hz, 1H), 5.03 (dd, J=10.0, 1.3 Hz, 1H), 4.03 (q, J=6.8 Hz, 1H), 3.79 (s, 3H), 3.77 (s, 3H), 3.25 (dq, J=9.5, 6.1 Hz, 2H), 3.09-3.01 (m, 1H), 2.88-2.74 (m, 3H), 2.37-2.28 (m, 1H), 1.48 (ddd, J=12.1, 12.1, 5.5 Hz, 1H), 1.38 (d, J=6.8 Hz, 3H).

### Example 8 Preparation of Compound 3a

A sealed vessel was charged with 4a (10 g, 34 mmoL), sodium carbonate (4.4 g, 41 mmoL), allyl bromide (4.4 g, 35 mmoL), sodium iodide (0.3 g, 1.7 mmoL) and acetonitrile (50 mL). The vessel was sealed under nitrogen and heated to 90° C. for 23.5 h. A portion of reaction solvent (20 mL) was then removed by distillation and the reaction was cooled to 22° C. The mixture was then extracted into ethyl acetate with saturated aqueous potassium carbonate solution and then brine solution followed by drying the organic phase over anhydrous sodium sulphate. The solids were removed by filtration and the mother liquor was concentrated to provide 3a as a yellow-orange viscous oil, 11 g, 95% yield, which was used directly without further purification.

### Example 9 Preparation of Compound 3a

A sealed vessel was charged with 4a (8.0 g, 26 mmoL), sodium carbonate (3.3 g, 31 mmoL), allyl bromide (3.2 g, 26 mmoL), sodium iodide (0.2 g, 1.3 mmoL) and 40 mL N,N-dimethylformamide. The vessel was sealed under nitrogen and heated to 90° C. for 24 h and then 110° C. for 17 h. Additional sodium iodide (0.2 g, 1.3 mmoL), sodium carbonate (1.6 g, 15 mmoL) and allyl bromide 1.6 g 14 mmoL) were charged to the reaction and the vessel was sealed and reheated to 110° C. for 3 h. The mixture was cooled to 22° C. and extracted into heptanes with saturated aqueous potassium carbonate solution and then brine solution, followed by drying the organic phase over anhydrous sodium sulphate. The solids were removed by filtration and the mother liquor was concentrated to provide 3a as a yellow-orange viscous oil, 8.5 g g, 94% yield, which was used directly without further purification.

### Example 10 Preparation of Compound 3b

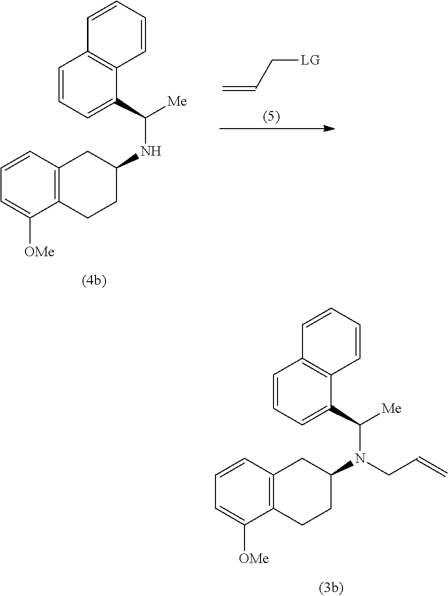

A sealed vessel was charged with 4b (0.43 g, 1.3 mmoL), sodium carbonate (0.28 g, 2.6 mmoL), allyl bromide (0.19 g, 1.6 mmoL) sodium iodide (0.21 g, 1.4 mmoL) and acetonitrile (4 mL). The vessel was sealed under nitrogen and heated to 95° C. for 17.5 h, at which point the reaction was determined to be complete by TLC. The reaction was then cooled to 22° C., filtered through Celite® to remove insoluble inorganics, and the mother liquor was concentrated to dryness. The residue was purified by silica gel column chromatography (gradient elution, 5-10% ethyl acetate in heptanes) to provide 3b as a clear, colourless viscous oil, 0.42 g, 87% yield. ¹H-NMR (400 MHz, CDCl₃): δ=8.41 (d, J=9.4 Hz, 1H), 7.80 (d, J=9.4 Hz, 1H), 7.69 (d, J=8.1 Hz, 1H), 7.58 (d, J=7.0 Hz, 1H), 7.46-7.37 (m, 3H), 7.05 (dd, J=7.8, 7.8 Hz, 1H), 6.69 (d, J=7.7 Hz, 1H), 6.60 (d, J=8.1 Hz, 1H), 5.95-5.85 (m, 1H), 5.12 (dd, J=17.0, 1.4 Hz, 1H), 4.96 (dd, J=10.7, 1.4 Hz, 1H), 4.83 (q, J=6.7 Hz, 1H), 3.75 (s, 3H), 3.41-3.31 (m, 2H), 3.13-3.06 (m, 1H), 3.00-2.94 (m, 2H), 2.23 (ddd, J=11.9, 11.4, 5.8 Hz, 1H), 1.88-1.83 (m, 1H), 1.64-0.155 (m, 1H), 1.54 (d, J=6.6 Hz, 3H).

### Example 11 Preparation of Compound 3b

A sealed vessel was charged with 4b (4.0 g, 12 mmoL), potassium carbonate (2.0 g, 15 mmoL), allyl bromide (1.7 g, 13 mmoL), potassium iodide (0.2 g, 1.2 mmoL) and 20 mL acetonitrile. The vessel was sealed under nitrogen and heated to 100° C. for 16.5 h at which point the reaction was determined to be complete by TLC. The reaction was cooled to 22° C. and then extracted into ethyl acetate with water, saturated aqueous potassium carbonate solution and then brine solution, followed by drying the organic phase over anhydrous sodium sulphate. The solids were removed by filtration and the mother liquor was concentrated to provide 3b as a yellow-orange viscous oil, 4.4 g, 99% yield, which was used directly without further purification.

### Example 12 Preparation of Compound 8a

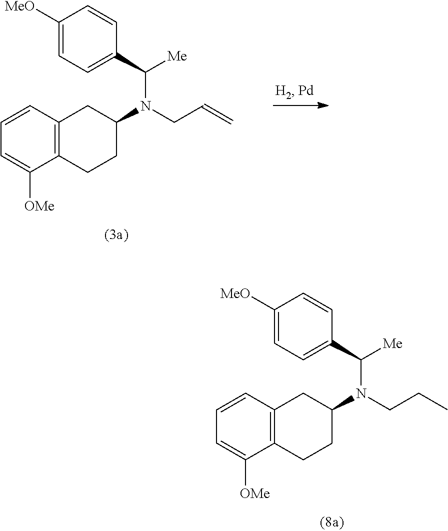 [SMILES: C=CCN([C@H]1CCc2c(cccc2OC)C1)[C@H](C)c1ccc(OC)cc1 ; CCCN([C@H]1CCc2c(cccc2OC)C1)[C@H](C)c1ccc(OC)cc1]

To a solution of 3a (4.4 g, 12 mmoL) in isopropanol (40 mL) was added a well shaken slurry of Raney™ Nickel 2800 in water (0.7 mL) under nitrogen atmosphere. The reaction was then placed under hydrogen atmosphere for 4 h at 22° C. Filtration through Celite®, concentration of the mother liquor and purification by silica gel column chromatography (10% ethyl acetate in heptanes) provided the reduced compound 8a as a clear and colourless oil, 3.4 g, 76% yield. ¹H-NMR (400 MHz, CDCl₃): δ=7.28 (d, J=8.7 Hz, 2H), 7.05 (dd, J=7.9, 7.8 Hz, 1H), 6.81 (d, J=8.8 Hz, 2H), 6.67 (d, J=7.7 Hz, 1H), 6.60 (d, J=8.1 Hz, 1H), 3.98 (q, J=6.8 Hz, 1H), 3.78 (s, 3H), 3.77 (s, 3H), 3.00-2.92 (m, 1H), 2.89-2.72 (m, 3H), 2.58-2.46 (m, 2H), 2.32 (ddd, J=11.8, 11.8, 5.8 Hz, 1H), 1.77-1.72 (m, 1H), 1.53-1.41 (m, 1H), 1.43 (q, J=7.5 Hz, 2H), 1.37 (d, J=6.8 Hz, 3H), 0.82 (t, J=7.5 Hz, 3H).

### Example 13 Preparation of Compound 8b

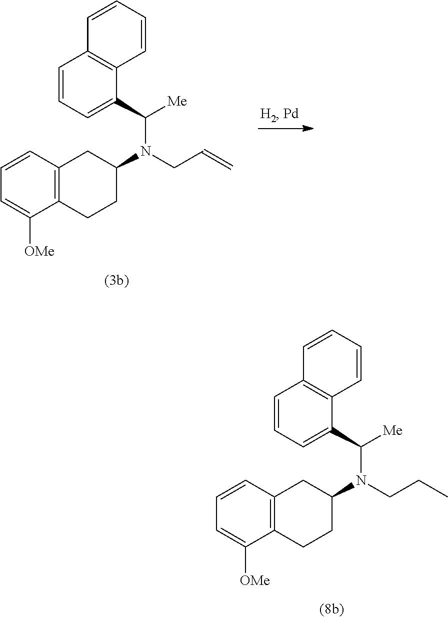 [SMILES: C=CCN([C@H]1CCc2c(cccc2OC)C1)[C@H](C)c1cccc2ccccc12 ; CCCN([C@H]1CCc2c(cccc2OC)C1)[C@H](C)c1cccc2ccccc12]

A solution of 3b (2.9 g, 7.8 mmoL) in heptanes (60 mL) was treated with 5% palladium on barium sulphate (0.15 g, 0.25 wt % Pd with respect to the compound 3b) to form a suspension which was stirred under a hydrogen atmosphere at 22° C. for 23 h. The reaction was filtered through Celite®, and the mother liquor was then concentrated. Purification by silica gel column chromatography (gradient elution, 0-2% ethyl acetate in heptanes) provided the reduced compound 8b as a clear and colourless oil, 2.8 g, 95% yield. ¹H-NMR (400 MHz, CDCl₃): δ=8.46 (d, J=9. Hz, 1H), 7.79 (dd, J=6.1, 2.4 Hz, 1H), 7.68 (d, J=8.1 Hz, 1H), 7.57 (d, J=7.1 Hz, 1H), 7.44-7.34 (m, 3H), 7.05 (dd, J=7.9, 7.9 Hz, 1H), 6.69 (d, J=7.7 Hz, 1H), 6.60 (d, J=8.0 Hz, 1H), 4.78 (q, J=6.7 Hz, 1H), 3.74 (s, 3H), 3.03-2.81 (m, 4H), 2.75-2.52 (m, 2H), 2.23 (ddd, J=11.7, 11.6, 6.1 Hz, 1H), 1.94-1.89 (m, 1H), 1.61-0.157 (m, 1H), 1.52 (d, J=6.8 Hz, 3H), 1.50-1.38 (m, 1H), 1.34-1.26 (m, 1H), 0.74 (t, J=7.2 Hz, 3H).

### Example 14 Preparation of Compound 2a

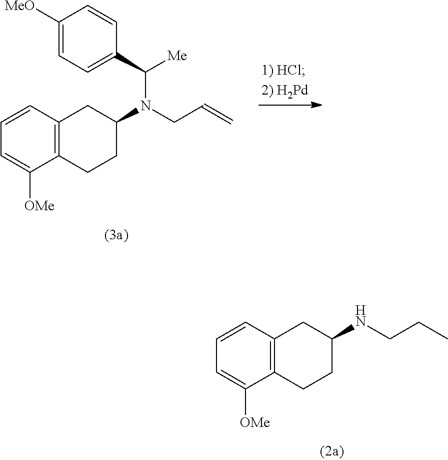 [SMILES: C=CCN([C@H]1CCc2c(cccc2OC)C1)[C@H](C)c1ccc(OC)cc1 ; CCCN[C@H]1CCc2c(cccc2OC)C1]

To a solution of 3a (0.78 g, 2.2 mmoL) in methyl t-butyl ether (5 mL) was added a solution of HCl in isopropanol (0.45 g, 2.2 mmoL, 18 wt % HCl in isopropanol). The resulting suspension and solution was concentrated in vacuo to give the HCl salt of 3a as a white tacky solid, which was used directly without further purification. The HCl salt of 3a (0.63 g 1.6 mmoL) was charged to an hydrogenation bomb reactor followed by 20% Pd(OH)₂/C/60% wetted (0.60 g, 0.6 wt % Pd with respect to the compound of 3a) and methanol (6 mL). The reactor was sealed under nitrogen, filled and vented 3 times with 100 psi nitrogen and then filled and vented 3 times with 120 psi hydrogen. The reaction was maintained at 120 psi hydrogen and 40° C. for 21 h. The reaction was then filtered through Celite®, washed with methanol and dichloromethane and the mother liquor was concentrated. The residue was extracted into dichloromethane and ethyl acetate with saturated aqueous potassium carbonate solution and then brine, and the organic layers were combined and concentrated. Purification by silica gel column chromatography (50% ethyl acetate in heptanes, 2% triethylamine) provided compound 2a free base as a clear and colourless oil, 0.16 g, 45% yield. ¹H-NMR (400 MHz, CDCl₃): δ=7.09 (dd, J=7.9, 7.9 Hz, 1H), 6.70 (d, J=7.6 Hz, 1H), 6.65 (d, J=8.1 Hz, 1H), 3.81 (s, 3H), 2.99 (dd, J=16.0, 4.4 Hz, 1H), 2.93-2.86 (m, 2H), 2.67 (dd, J=7.4, 7.4 Hz, 2H), 2.61-2.52 (m, 2H), 2.11-2.05 (m, 1H), 1.60-1.47 (m, 3H), 0.94 (t, J=7.4 Hz, 3H).

### Example 15 Preparation of Compound 2a

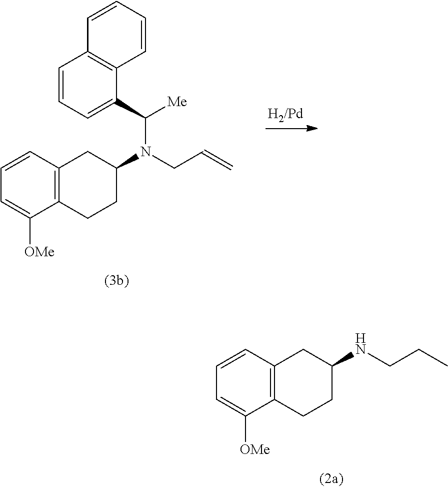 [SMILES: C=CCN([C@H]1CCc2c(cccc2OC)C1)[C@H](C)c1cccc2ccccc12 ; CCCN[C@H]1CCc2c(cccc2OC)C1]

Compound 3b (0.49 g 1.3 mmoL) was charged in a 1:1 mixture of acetic acid:dichloromethane (6 mL) followed by 20% Pd(OH)₂/C/60% wetted (0.50 g, 0.82 wt % Pd with respect to the compound 3b). The reactor was sealed under nitrogen, filled and vented 3 times with 100 psi nitrogen and then filled and vented 3 times with 120 psi hydrogen. The reaction was maintained at 120 psi hydrogen and 60° C. for 19.5 h. The reaction was then filtered through Celite®, washed with dichloromethane and the mother liquor was concentrated and then extracted into ethyl acetate with saturated aqueous potassium carbonate solution, distilled water and then brine, and then dried over anhydrous sodium sulphate. The solids were removed by filtration and the mother liquor was concentrated. Purification by silica gel column chromatography (50% ethyl acetate in heptanes, 2% triethylamine) provided compound 2a free base as a clear and colourless oil, 0.07 g, 24% yield.

### Example 16 Preparation of Compound 2a

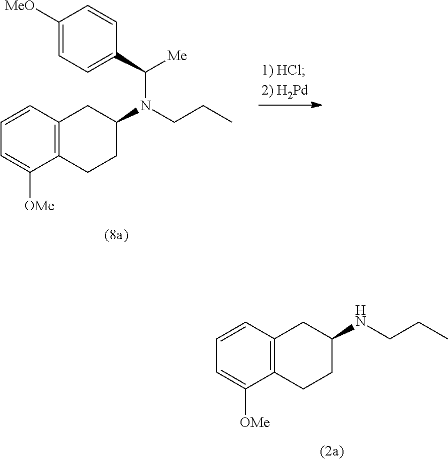 [SMILES: CCCN([C@H]1CCc2c(cccc2OC)C1)[C@H](C)c1ccc(OC)cc1 ; CCCN[C@H]1CCc2c(cccc2OC)C1]

An hydrogenation bomb reactor was charged with 8a (1.0 g 2.8 mmoL) 10 Pd/C/50% wetted (0.5 g, 0.25 wt % Pd with respect to the compound 8a) a mixture of isopropanol:distilled water, (1:1 v/v, 6 mL) and a solution of HCl in isopropanol (0.60 g, 3.0 mmoL, 18 wt % HCl in isopropanol). The reactor was sealed under nitrogen, filled and vented 3 times with 100 psi nitrogen and then filled and vented 3 times with 120 psi hydrogen. The reaction was maintained at 120 psi hydrogen and 40° C. for 22 h. The reaction was neutralized with 5N sodium hydroxide, diluted with ethyl acetate (30 mL) and then filtered through Celite®. The organic phase of the mother liquor was separated and concentrated to 12 mL total volume. A solution of HCl in isopropanol (0.60 g, 3.0 mmoL, 18 wt % HCl in isopropanol) was added and the slurry was further diluted with ethyl acetate. The precipitate was collected by filtration, washed with excess ethyl acetate and then dried under vacuum at room temperature to provide 2a HCl salt as a white solid, 0.56 g, 77% yield.

### Example 17 Preparation of Compound 2a

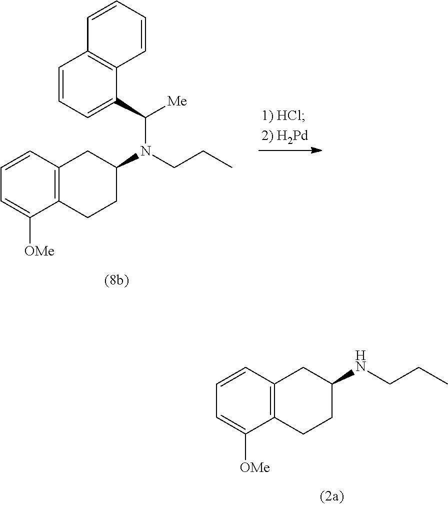 [SMILES: CCCN([C@H]1CCc2c(cccc2OC)C1)[C@H](C)c1cccc2ccccc12 ; CCCN[C@H]1CCc2c(cccc2OC)C1]

An hydrogenation bomb reactor was charged with 8b (1.8 g 4.8 mmoL) 10% Pd/C/50% wetted (0.17 g, 0.47% Pd with respect to the compound 8b) a mixture of isopropanol:distilled water, (2:1 v/v, 20 mL) and a solution of HCl in isopropanol (0.98 g, 4.8 mmoL, 18 wt % HCl in isopropanol). The reactor was sealed under nitrogen, filled and vented 3 times with 100 psi nitrogen and then filled and vented 3 times with 120 psi hydrogen. The reaction was maintained at 120 psi hydrogen and 40° C. for 23 h. The reaction was diluted with methanol (20 mL) and then basified with 5N sodium hydroxide (2 mL) and then filtered through Celite®. The mother liquor was concentrated and then extracted into ethyl acetate with saturated aqueous potassium carbonate solution, distilled water and then brine and then concentrated in vacuo. The resultant crude oily residue was diluted and concentrated 3 times with isopropanol (10 mL) followed by dilution with isopropanol (10 mL). The solution was cooled to 0-5° C. and a solution of HCl in isopropanol (0.83 g, 4.8 mmoL, 21 wt % HCl in isopropanol) was added. The slurry was stirred at 0-5° C. for 2 h. The precipitate was collected by filtration, washed with isopropanol (2 mL) and then dried under vacuum at room temperature to provide 2a HCl salt as a white solid, 1.02 g, 83% yield.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

1. A process for the preparation of a compound of Formula (2): 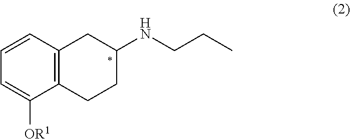
or a salt thereof, the process comprising hydrogenating, in the presence of a catalyst, a compound of Formula (3): 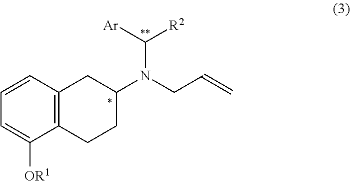
wherein
R¹ is selected from the group consisting of: H, C₁-C₃ alkyl, and 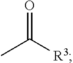
R² is selected from the group consisting of: H and C₁-C₃ alkyl;
R³ is selected from the group consisting of: C₁-C₆ alkyl, C₆-C₁₀ aryl and C₇-C₂₀ arylalkyl;
Ar is selected from the group consisting of: Ar-aryl and substituted Ar-aryl;
the carbon atom marked with “*” is racemic, enantiomerically enriched in the (R)-configuration, or enantiomerically enriched in the (S)-configuration; and
when R² is not H, the carbon atom marked with “**” is racemic, enantiomerically enriched in the (R)-configuration, or enantiomerically enriched in the (S)-configuration.

2. The process of claim 1, wherein the catalyst is selected from the group consisting of: palladium, platinum and Raney™ Nickel.

3. The process of claim 1, wherein the catalyst is selected from the group consisting of: palladium hydroxide on carbon and palladium on carbon.

4. The process of claim 3, further comprising isolating an intermediate compound of Formula (8): 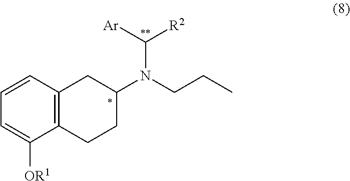
or a salt thereof during the hydrogenating.

5. The process of claim 1, further comprising reacting a compound of Formula (4): 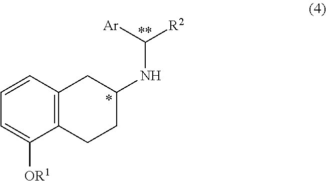
with a compound of Formula (5): 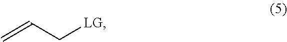
thereby forming the compound of Formula (3), wherein
LG is a leaving group.

6. The process of claim 5, wherein the leaving group is selected from the group consisting of: bromide, iodide, sulfonyloxy groups and carbonates.

7. The process of claim 5, further comprising reductive amination of a compound of Formula (6): 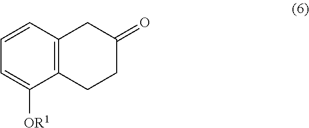
with a compound of Formula (7): 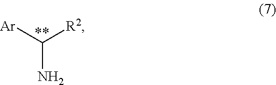
thereby forming the compound of Formula (4).

8. The process of claim 7, further comprising isolating an intermediate compound of Formula (9): 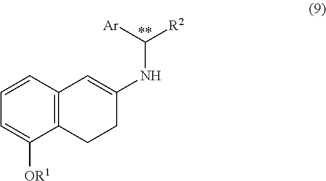
or a salt thereof.

9. The process of claim 7, wherein the reductive amination is conducted with a hydride reducing agent selected from the group consisting of: sodium borohydride, potassium borohydride, lithium borohydride, sodium cyanoborohydride and sodium triacetoxyborohydride.

10. The process of claim 1, wherein
R¹ is C₁-C₃ alkyl;
R² is methyl;
Ar is selected from the group consisting of: naphthyl, phenyl, and substituted phenyl;
the carbon atom marked with “*” is enriched in the (S)-configuration; and
the carbon atom marked with “**” is enriched in the (R)-configuration.

11. The process of claim 10, wherein the compound of Formula (3) is selected from the group consisting of: 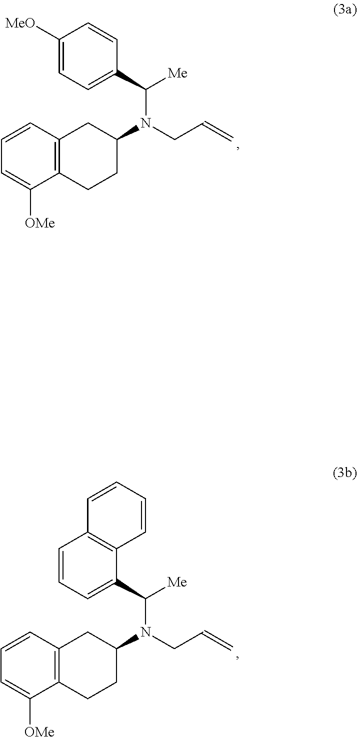 [SMILES: C=CCN([C@H]1CCc2c(cccc2OC)C1)[C@H](C)c1ccc(OC)cc1 ; C=CCN([C@H]1CCc2c(cccc2OC)C1)[C@H](C)c1cccc2ccccc12]
and salts thereof.

12. The process of claim 6, wherein the compound of: Formula (4) or salt thereof is prepared by the reductive amination of a compound of Formula (6): 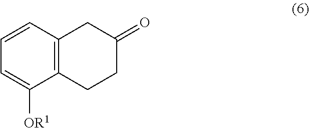
with a compound of Formula (7): 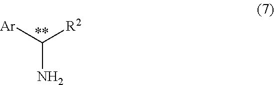
wherein
R¹ is selected from the group consisting of: H, C₁-C₃ alkyl, and 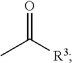
R² is C₁-C₃ alkyl;
R³ is selected from the group consisting of: C₁-C₆ alkyl, C₆-C₁₀ aryl and C₇-C₂₀ arylalkyl;
Ar is selected from the group consisting of: naphthyl and substituted phenyl;
the carbon atom marked with “*” is enantiomerically enriched in the (R)-configuration, or enantiomerically enriched in the (S)-configuration; and
the carbon atom marked with “**” is enantiomerically enriched in the (R)-configuration, or enantiomerically enriched in the (S)-configuration.

13. The process of claim 12, further comprising isolating an intermediate compound of Formula (9): 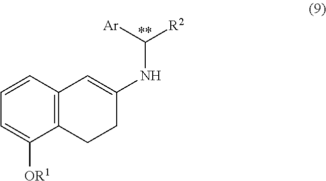
or a salt thereof.

14. The process of claim 12, wherein the reductive amination is conducted with a hydride reducing agent selected from the group consisting of: sodium borohydride, potassium borohydride, lithium borohydride, sodium cyanoborohydride and sodium triacetoxyborohydride.

15. The process of claim 12, wherein the compound of Formula (4) is selected from the group consisting of: 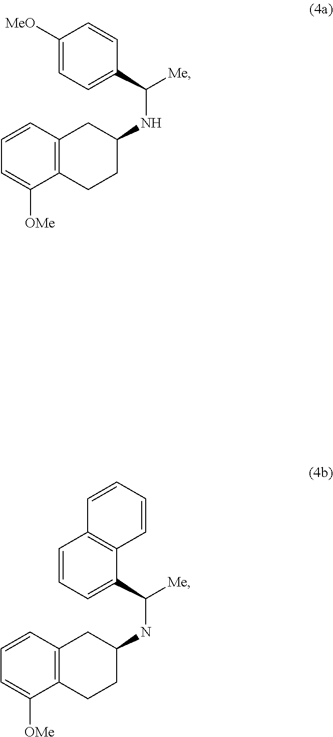 [SMILES: COc1ccc([C@@H](C)N[C@H]2CCc3c(cccc3OC)C2)cc1 ; COc1cccc2c1CC[C@H](N[C@H](C)c1cccc3ccccc13)C2]
and salts thereof.

16. A compound of Formula (3): 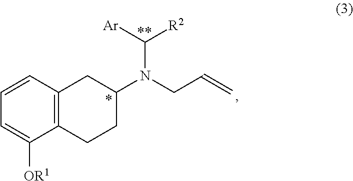
or a salt thereof
wherein
R¹ is selected from the group consisting of: H, C₁-C₃ alkyl,
and 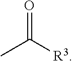
R² is selected from the group consisting of: H and C₁-C₃ alkyl;
R³ is selected from the group consisting of: C₁-C₆ alkyl, C₆-C₁₀ aryl and C₇-C₂₀ arylalkyl;
Ar is selected from the group consisting of: Ar-aryl and substituted Ar-aryl;
the carbon atom marked with “*” is racemic, enantiomerically enriched in the (R)-configuration, or enantiomerically enriched in the (S)-configuration; and
when R² is not H, the carbon atom marked with “**” is racemic, enantiomerically enriched in the (R)-configuration, or enantiomerically enriched in the (S)-configuration.

17. The compound of claim 16, wherein the compound of Formula (3) is a compound selected from the group consisting of: 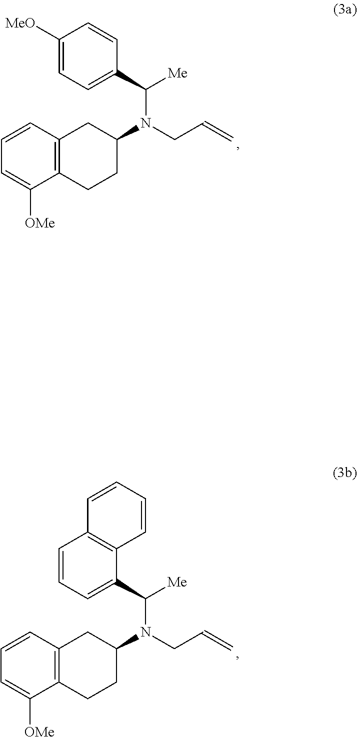 [SMILES: C=CCN([C@H]1CCc2c(cccc2OC)C1)[C@H](C)c1ccc(OC)cc1 ; C=CCN([C@H]1CCc2c(cccc2OC)C1)[C@H](C)c1cccc2ccccc12]
and salts thereof.

18. (canceled)

19. A compound selected from the group consisting of: 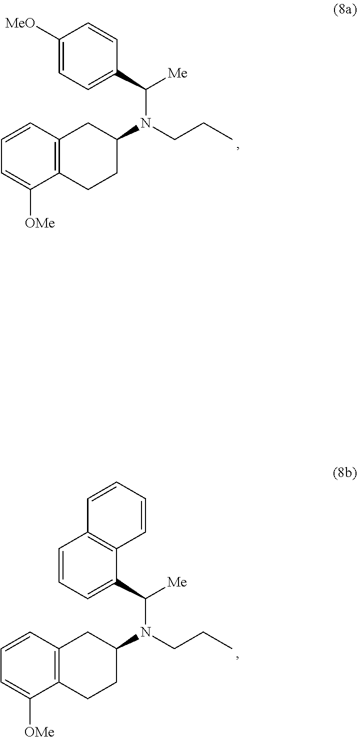 [SMILES: CCCN([C@H]1CCc2c(cccc2OC)C1)[C@H](C)c1ccc(OC)cc1 ; CCCN([C@H]1CCc2c(cccc2OC)C1)[C@H](C)c1cccc2ccccc12]
and salts thereof.

20. (canceled)

21. The process of claim 1, further comprising converting the compound of Formula (2) or a salt thereof to Rotigotine or a pharmaceutically acceptable salt thereof.
